(19) United States
(12) Patent Application Publication
(10) Pub. No.: US 2008/0119942 A1
(43) Pub. Date: May 22, 2008

(54) Short stem femoral prosthesis

(75) Inventors: Mario Mercuri, Bologna (IT); Francesco Falez, Rome (IT); Enrico Vaienti, Parma (IT); Leo Massari, Ferrara (IT); Francesco Benazzo, Pavia (IT); Alessandro Masse, Torino (IT); Carlo Ruosi, Napoli (IT); Massimiliano Marcucci, Firenze (IT)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/803,934

(22) Filed: May 15, 2007

(30) Foreign Application Priority Data
Nov. 16, 2006 (GB) 0622930.6

(51) Int. Cl. A61F 2/32; A61F 2/30

(52) U.S. Cl. 623/22.11; 623/23.44

(57) ABSTRACT

A prosthetic hip implant comprising: a neck portion including a prosthetic head mounted thereon; and a stem portion coupled to the neck portion having a generally u-shaped cross-section, the stem curving towards a medial side of a femur on moving in a proximal to distal direction when the stem is implanted, a base portion of the u-shaped cross-section extending between first and second legs of the u-shaped cross-section, the base portion decreasing in thickness while moving in a proximal to distal direction and a length of the first and second legs of the u-shaped cross-section decreasing on moving in a proximal to distal direction.

### BACKGROUND OF THE INVENTION

This invention relates to a short stem prosthesis.

Femoral prostheses are known which have a stem which, when fitted in a suitably prepared femur from which the head and at least part of the shoulder have been removed, extend downwards past the lesser trochanter and into the medullary canal in the shaft of the bone. Such stems can be fixed into the canal of the bone during implantation by utilizing their tapered shape by cementing or other means. Prostheses of this type are well known.

Traditional neck filling stems of this type can require the removal of a large amount of trabecular bone. Stress shielding can be expected and the consequent reabsorption of the neck can endanger the stability of the stem.

The present invention is intended to provide a construction which joints the concept of a proximal stem placed in the neck of the bone and following the anatomy of the boney travecular (bone sparing) and provide a design which aims to avoid the pitfalls which have occurred in former designs.

### SUMMARY OF THE INVENTION

The stem of the present invention only extends for a short distance into the intramedullary canal and it is mainly located in a portion of the neck of the bone. A prosthesis employing this type of stem will therefore be referred to herein as a “short” femoral prosthesis, i.e. one which extends down the lesser trochanter but not into the diaphysis (shaft of the bone). Thus, its fixation is in the metaphysics. The length of the stem of such a “short” prosthesis for a medium size femur would be about 60 mm.

According to the present invention, a one-piece short femoral prosthesis comprises a proximal end portion having, or adapted to receive, a bearing ball head and a gutter-shaped or u-shaped stem having anterior and posterior sides which extends from the proximal end portion to an open distal end curved in a medial/lateral plane through its length and closed by a substantially radially extending proximal end wall adjacent said proximal end portion.

It is believed that a stem of this shape has the advantage to respect and spare the tractive trabecular system in the supero-lateral part of the femur. This portion is usually referred to as the principle tensile group and is usually not spared, even with the use of conservative standard long stem hip implants.

The gutter or u-shape allows the introduction of the stem with the minimal removal of bone, maximum rotational stability, full support from the bone, loose travacular will oppose the bending forces, large bone/metal interface, minimally invasive instruments which also help in terms of bone sparing. The gutter of unshaped groove along the profile of the stem can increase by 20% the surface area in connect with bone, thereby increasing the potential for boney in-growth onto the larger surface area.

In a preferred embodiment the gutter shaped stem has a medial side which is formed as a curve of constant radius from a first centre point and a lateral side which is also formed as a constant radius curve from a second centre point so that the stem tapers from its proximal end to its distal end. In one preferred embodiment the gutter-shaped stem has an outer surface and an inner gutter surface which are both substantially square or rectangular in cross-section.

In yet another embodiment the u-shaped stem can have an outer surface which is part-circular, square or rectangular and an inner surface which has a semicircular lower portion and extended upper side walls in cross-section. In any of the above constructions the gutter can be provided in the medial side of the stem or, alternatively, on the lateral side of the stem. If desired, the wall thickness of the gutter may be arranged to decrease proximally to distally. The proximal end of the stem may be provided with a projecting flange or collar.

In the preferred embodiment the proximal end portion is provided with an integral conically tapered boss or trunion to receive a cooperating modular ball head.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

FIG. 1 is a side elevation of a one-piece “short” femoral prosthesis, according to the invention in which the gutter-shaped stem is of substantially square or rectangular cross-section and in which the gutter is provided on the lateral side of the stem;

FIG. 2 is a cross-sectional side elevation of the prosthesis shown in FIG. 1;

FIG. 3 is an isometric view from above of the prosthesis shown in FIGS. 1 and 2;

FIG. 4 is a cross-sectional side elevation of an alternative construction in which the cross-section of the gutter is substantially square or rectangular and the gutter is provided on the medial side of the stem;

FIG. 5 is an end view of the gutter shown in FIG. 4;

FIG. 6 is an isometric view from above of the gutter shown in FIGS. 4 and 5;

FIG. 7 is a side elevation of a prosthesis, according to the present invention, in which the gutter is of substantially circular cross-section and is on the lateral side of the stem;

FIG. 8 is a cross-sectional view of the construction shown in FIG. 7;

FIG. 9 is an isometric view from above of the gutter shown in FIGS. 7 and 8;

FIG. 10 is a part isometric view from below of the prosthesis shown in FIGS. 7, 8 and 9;

FIG. 11 is a side elevation of a prosthesis, according to the invention, in which the gutter is substantially part-circular and is located on the medial side of the stem;

FIG. 12 is a cross-sectional side elevation of the prosthesis shown in FIG. 11;

FIG. 13 is an isometric view from above of the gutter shown in FIGS. 11 and 12;

FIG. 14 is an isometric view from below of the prosthesis shown in FIGS. 11, 12 and 13;

FIG. 15 is a plan view from above of a prosthesis according to the invention in which the outer surface of the stem is part-circular and the gutter surface has a semicircular lower portion and extended upper side walls in cross-section and also shows how a flange or collar can be provided;

FIG. 16 is a cross-sectional side elevation of the prosthesis shown in FIG. 15;

FIG. 17 is an end view of the prosthesis shown in FIGS. 15 and 16;

FIG. 18 is an isometric view from above of the construction shown in FIGS. 15, 16 and 17, and

FIG. 19 is an isometric view from below of the construction shown in FIGS. 15, 16, 17 and 18.

### DETAILED DESCRIPTION

As shown in FIGS. 1, 2 and 3 a one-piece “short” femoral prosthesis 1 comprises a proximal end portion 2 which, in this embodiment, is provided with a tapered boss 3 to receive a cooperating bearing ball 4 shown in broken lines. In an alternative construction the bearing ball could be integral with the proximal end portion 2.

A gutter-shaped or U-shaped hollow stem 5 is provide which has anterior and posterior sides or legs 6 and 7 and a medial base 11, respectively, which extend from the proximal end portion 2 to an open distal end 8. The gutter shaped stem 5 is curved in a medial/lateral plane throughout its length and is closed by a substantially radially extending proximal end wall 9 adjacent the proximal end portion 2. The legs 6, 7 can be angled outwardly toward the open end of the U-shaped cross-section, for example, by 8 to 14: This angle can vary along the stem length.

As will be seen, an open topped gutter 10 is provided in the stem 5 and in this construction is in the lateral side of the stem.

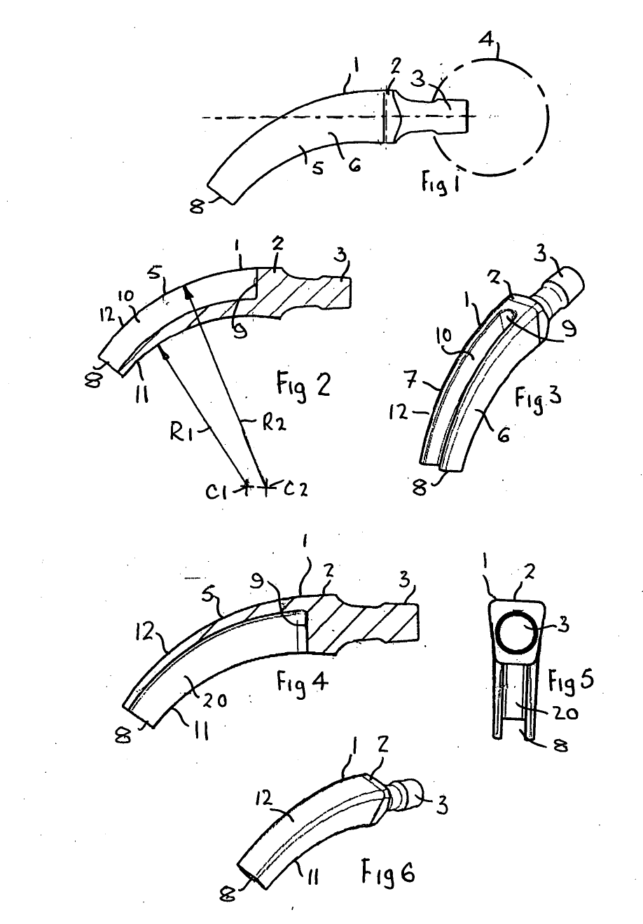

The gutter shaped stem 5 medial side 11 is formed as a curve of constant radius R1 from a first center point C1. The lateral side 12 is also formed as a constant radius, indicated by arrow R2, from a center point C2 spaced away from the center portion C1 so that the stem 5 tapers from its proximal end at the proximal end portion 2 to its distal open end 8 as shown in FIG. 2. The wall thickness of the gutter decreases proximally to distally.

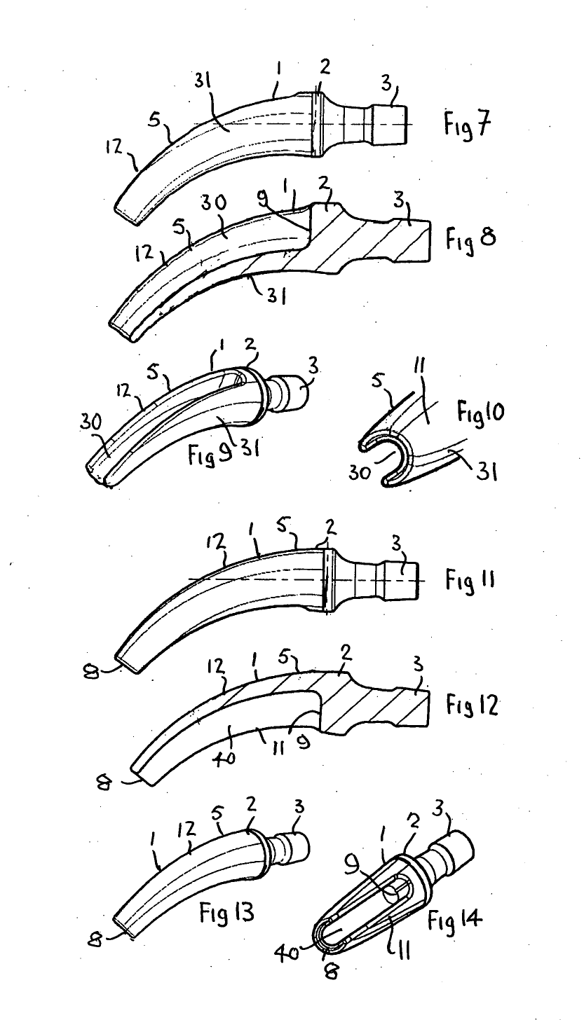

FIG. 4 shows an embodiment similar to that shown in FIGS. 1, 2 and 3 and similar reference numerals are used to indicate similar parts. In this construction the gutter 20 is inverted to that shown in FIGS. 1, 2 and 3 and faces downwardly and is provided on the medial side 11 of the stem 5. The cross-sectional shape of the gutter is again substantially square or rectangular.

FIGS. 7, 8, 9 and 10 show another embodiment in accordance with the invention in which the same reference numerals are used to indicate similar parts to those shown in FIGS. 1, 2 and 3. The general construction is similar but the cross-section of the gutter 30 is part-circular, as is the outer surface 31. The gutter is provided on the lateral side 12 of the stem.

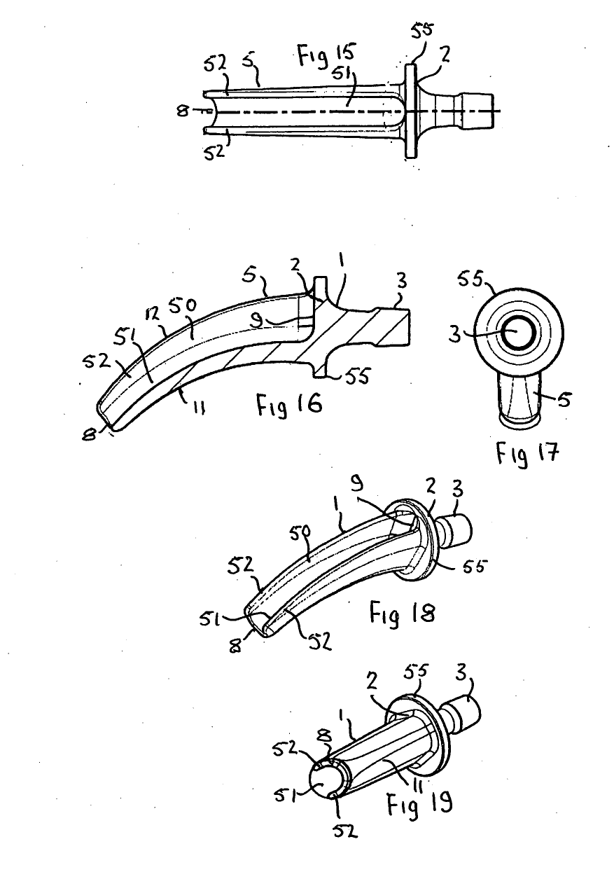

In the embodiment shown in FIGS. 11, 12, 13 and 14 the general configuration and shape is similar to that shown in FIGS. 7, 8, 9 and 10, inasmuch that the gutter and outer surface are both part-circular but in this arrangement the gutter 40 is provided on the medial side 11 of the stem 5. Again the wall thickness of the gutter decreases proximally to distally and the curve of the stem is similar to that described with regard to FIG. 2.

FIGS. 15, 16, 17, 18 and 19 show another embodiment which is similar in general shape to that described with regard to FIGS. 1, 2 and 3 and the same reference numerals are used to indicate similar parts but in this construction the gutter-shaped stem 5 has an outer surface which is part-circular and has a gutter 50 with a surface which has a lower semicircular portion 51 and extended upper side walls 52. With this type of construction the outer surface could alternatively be part square or rectangular, as shown in FIGS. 1, 2 and 3.

The curve of the stem is similar to that shown in the other construction. As shown in FIGS. 15, 16, 17, 18 and 19 the gutter is on the lateral side 12 of the stem 5 but it could be alternatively provided on the medial side 11, as shown in the constructions illustrated in FIGS. 4 to 6 and 7 to 10.

FIGS. 16, 17, 18 and 19 also show how a flange or collar 55 can be provided on the proximal end portion 2 of the stem if required for a particular type of surgery.

Collars similar to the collar 55 could be provided on any of the other constructions described above.

For a medium size femur of approximately 20-255 mm the length of stem 5 in the bone will be about 50 to 60 mm for a medium size stem. The width of the stem at its closed end may be about 12 mm.

The prosthesis can be made as a one-piece construction from any suitable metal material, for example titanium or stainless steel and shaped by, for example, molding, casting, forging or any other process.

If desired the surface of the stem can be prepared to facilitate fixture, for example, by cement, or to assist bone growth.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic hip implant comprising:
a neck portion including a prosthetic head mounted thereon; and
a stem portion coupled to the neck portion having a generally u-shaped hollow cross-section, the stem curving towards a medial side of a femur on moving in a proximal to distal direction when the stem is implanted, a base portion of the u-shaped cross-section extending between first and second legs of the u-shaped cross-section, the base portion decreasing in thickness while moving in a proximal to distal direction and a length of the first and second legs of the u-shaped cross-section decreasing on moving in a proximal to distal direction.

2. The prosthetic hip implant as set forth in claim 1 wherein the base portion of the u-shaped cross-section is perpendicular to the first and second legs.

3. The prosthetic implant as set forth in claim 1 wherein the length of the stem is about 60 mm.

4. The prosthetic implant as set forth in claim 1 wherein the u-shape cross-section is open at a distal end of the stem.

5. The hip implant as set forth in claim 1 wherein the base portion is arcuate.

6. The hip implant as set forth in claim 1 wherein the u-shaped stem cross-section is open in a medial or a lateral direction.

7. The hip implant as set forth in claim 1 wherein the first and second legs of the unshaped cross-section are spaced a constant anterior-posterior on moving from the proximal to distal direction.

8. The prosthetic hip implant as set forth in claim 1 wherein the u-shaped cross-section of the stem has a closed proximal end wall adjacent the neck portion.

9. The prosthetic hip implant as set forth in claim 1 wherein the stem has a medial and a lateral side which lie along an arc of a circle.

10. The prosthetic hip implant as set forth in claim 9 wherein the stem has a medial and a lateral side which lie along an arc of a circle.

11. The prosthetic hip implant as set forth in claim 10 wherein the centers of the circular arcs of the medial and lateral sides of the hip implant lie in the same plane and are off-set such that the medial and lateral sides converge while moving in a proximal to distal direction.

12. A prosthetic hip implant comprising:
a stem portion for insertion into the proximal femur, the stem having a cross-section with a base and first and second legs extending from opposite sides of the base forming a hollow u-shaped open on a side opposite the base, the base decreasing in thickness on moving from a proximal end of the stem to a distal end.

13. The prosthetic hip implant as set forth in claim 12 wherein a length of the first and second legs decreases on moving from the proximal to the distal end of the stem.

14. The prosthetic hip implant as set forth in claim 13 wherein the base portion of the u-shaped cross-section is perpendicular to the first and second legs.

15. The prosthetic implant as set forth in claim 14 wherein the u-shaped cross-section is open at a distal end of the stem.

16. The hip implant as set forth in claim 15 wherein the base portion is arcuate.

17. The hip implant as set forth in claim 16 wherein the u-shaped stem cross-section is open in a medial or a lateral direction.

18. The hip implant as set forth in claim 17 wherein the first and second legs of the u-shaped cross-section are spaced a constant anterior-posterior on moving from the proximal to distal direction.

19. The prosthetic hip implant as set forth in claim 18 wherein the stem has a medial and a lateral side which lie along an arc of a circle.

20. The prosthetic hip implant as set forth in claim 19 wherein the centers of the circular arcs of the medial and lateral sides of the hip implant lie in the same plane and are off-set such that the medial and lateral sides converge while moving in a proximal to distal direction.
